(19) United States
(12) Patent Application Publication
(10) Pub. No.: US 2026/0165670 A1
(43) Pub. Date: Jun. 18, 2026

(54) X-RAY COMPUTED TOMOGRAPHY APPARATUS AND INFORMATION PROCESSING METHOD

(71) Applicant: CANON MEDICAL SYSTEMS CORPORATION, Otawara-shi (JP)

(72) Inventor: Ryusei SAIKI, Nasushiobara (JP)

(73) Assignee: CANON MEDICAL SYSTEMS CORPORATION, Otawara-shi (JP)

(21) Appl. No.: 19/413,115

(22) Filed: Dec. 9, 2025

(30) Foreign Application Priority Data
Dec. 13, 2024 (JP) 2024-218780

(51) Int. Cl. A61B 6/00; A61B 6/03; A61B 6/50

(52) U.S. Cl. CPC A61B 6/544; A61B 6/032; A61B 6/504

(57) ABSTRACT

An X-ray computed tomography (CT) apparatus according to an embodiment includes processing circuitry configured to identify gantry state information indicating a state of a gantry including an opening, the gantry state information including at least one of posture information indicating a posture of the gantry and object information indicating an object other than a subject present in the opening, determine a VolumeEC parameter used for calculation processing related to VolumeEC, which is a function to adjust X-ray irradiation conditions for the subject, based on the identified gantry state information and parameter information to be stored by associating the gantry state information with the VolumeEC parameter for calculation processing related to the VolumeEC, perform calculation processing related to the VolumeEC using the determined VolumeEC parameter, and adjust the X-ray irradiation conditions based on a result of the calculation processing related to the VolumeEC.

### CROSS-REFERENCE TO RELATED APPLICATIONS

This application is based upon and claims the benefit of priority from Japanese Patent Application No. 2024-218780, filed Dec. 13, 2024, the entire contents of which are incorporated herein by reference.

### FIELD

Embodiments described herein relate to an X-ray computed tomography (CT) apparatus and an information processing method.

### BACKGROUND

An X-ray computed tomography (CT) apparatus configured to capture an image of a subject in a lying-position state or a standing-position state has heretofore been known.

An X-ray CT apparatus having a VolumeEC function for adjusting an X-ray intensity so as to obtain an image with a designated image quality based on information about the body thickness of a subject, a CT value, and the like estimated based on a scanogram of the subject as a function for reducing an exposure does of the subject is known. For example, the use of VolumeEC makes it possible to capture an image of a subject using minimum X-ray irradiation conditions under which high image quality can be secured while reducing noise.

In an X-ray CT apparatus configured to capture images of a subject in a lying position state and a standing position state, a couch (couchtop) is generally used to support the subject in the lying position state, and a columnar subject holder (also referred to as a subject fixing pole) is generally used to support the subject in the standing position state.

Thus, an object other than the subject that is present in an opening of a gantry during imaging in a lying-position mode is different from an object other than the subject that is present in the opening of the gantry during imaging in a standing-position mode (or a seated-position mode), to be more specific, the couchtop is present in the opening of the gantry during imaging in the lying-position mode, while the subject fixing pole is present in the opening of the gantry during imaging in the standing-position mode (or the seated-position mode). For this reason, the amount of absorption of X-rays in the subject during imaging in the lying-position mode is different from that during imaging in the standing-position mode.

For this reason, there may be a difference in the quality of an image to be generated depending on the imaging mode even in a case where the same imaging conditions are used in the same X-ray CT apparatus. For example, in the case of capturing a scanogram of a subject, even when the same imaging conditions are used in the same X-ray CT apparatus, the image quality of the scanogram may vary depending on which one of the lying-position mode and the standing-position mode is set as an imaging mode.

In the VolumeEC function described above, the body thickness of a subject, a CT value, and the like are estimated based on a scanogram of the subject. In other words, there is a possibility that the body thickness of the subject, the CT value, and the like cannot be accurately estimated and X-ray irradiation conditions for obtaining the CT image with a designated image quality cannot be accurately derived depending on the imaging mode.

### BRIEF DESCRIPTION OF THE DRAWINGS

FIG. 1 illustrates a configuration example of an X-ray computed tomography (CT) apparatus according to an embodiment;

FIG. 2 is a perspective view illustrating an example of a state of a gantry apparatus in a standing-position mode according to the embodiment;

FIG. 3 is a perspective view illustrating an example of the state of the gantry apparatus in a lying-position mode according to the embodiment;

FIG. 4 illustrates an example of a scanogram of a subject captured in the lying-position mode according to the embodiment;

FIG. 5 illustrates an example of the scanogram of the subject captured in the standing-position mode according to the embodiment; and

FIG. 6 is a flowchart illustrating an example of processing to be executed by the X-ray CT apparatus according to the embodiment.

### DETAILED DESCRIPTION

An X-ray computed tomography (CT) apparatus according to an embodiment of the present disclosure includes an identification unit, a determination unit, a calculation unit, and an adjustment unit. The identification unit identifies gantry state information indicating a state of a gantry including an opening. The gantry state information includes at least one of posture information indicating a posture of the gantry, and object information indicating an object other than a subject present in the opening. The determination unit determines a VolumeEC parameter used for calculation processing related to VolumeEC, which is a function to adjust X-ray irradiation conditions for the subject, based on the identified gantry state information and parameter information to be stored by associating the gantry state information with the VolumeEC parameter for calculation processing related to the VolumeEC. The calculation unit performs calculation processing related to VolumeEC using the determined VolumeEC parameter. The adjustment unit adjusts the X-ray irradiation conditions based on a result of the calculation processing related to VolumeEC.

Various Embodiments will be described hereinafter with reference to the accompanying drawings.

An X-ray CT imaging apparatus and an information processing method according to embodiments will be described below with reference to the drawings. The X-ray CT apparatus according to the present embodiment has a structure in which the posture of the gantry can be changed between a standing-position imaging state in which an image of a subject P can be captured in a standing position and a lying-position imaging state in which an image of the subject P can be captured in a lying position. In the following embodiments, assuming that components denoted by the same reference numerals perform similar operations, redundant descriptions will be omitted as appropriate.

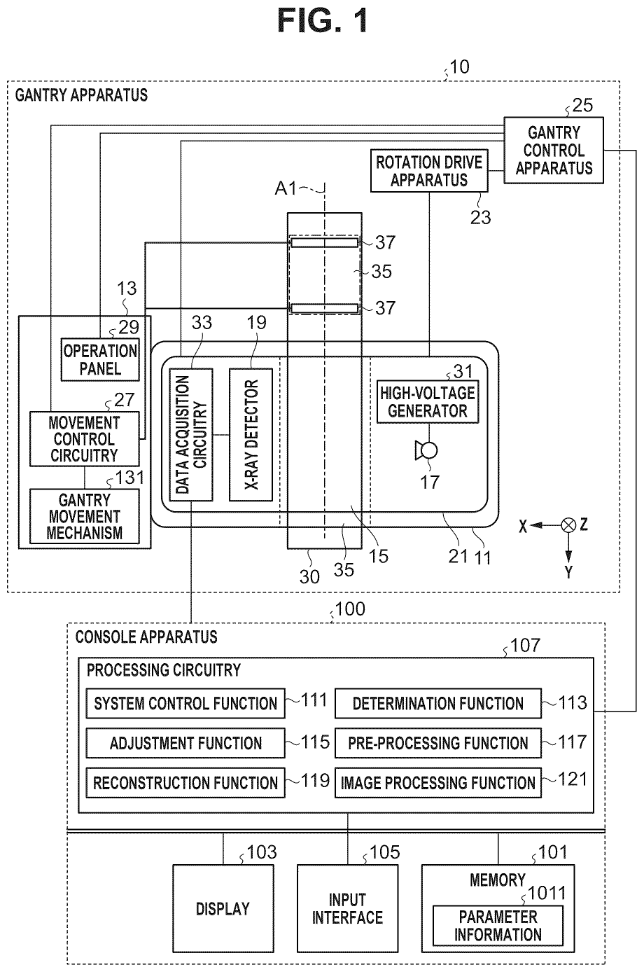

FIG. 1 illustrates a configuration example of an X-ray CT apparatus 1 according to an embodiment. As illustrated in FIG. 1, the X-ray CT apparatus 1 includes a gantry apparatus 10 and a console apparatus 100. For example, the gantry apparatus 10 is installed in a CT examination room, and the console apparatus 100 is installed in a control room that is adjacent to the CT examination room. The gantry apparatus 10 and the console apparatus 100 are connected by a wire or wirelessly so that the gantry apparatus 10 and the console apparatus 100 can communicate with each other.

In the present embodiment, an axial direction perpendicular to a floor surface, that is, a vertical direction, is defined as a Z-axis direction, and two directions that are perpendicular to the Z-axis direction and are perpendicular to each other are defined as an X-axis direction and a Y-axis direction, respectively.

The gantry apparatus 10 is a scanning apparatus configured to perform X-ray CT imaging on the subject P in a standing position state or a lying position state. The console apparatus 100 is a computer that controls the gantry apparatus 10.

The gantry apparatus 10 includes a gantry 11, a post 13, a rotation drive apparatus 23, and a gantry control apparatus 25.

The gantry 11 includes an imaging system related to imaging of the subject P, and an opening 15 into which the subject P can be inserted. The post 13 supports the gantry 11 so that the orientation of the opening 15 can be changed between the vertical direction and the horizontal direction and the gantry 11 can be moved along the vertical direction.

While FIG. 1 illustrates an example where the gantry 11 is supported in a cantilever manner by the post 13, the present embodiment is not limited to this example. For example, the gantry 11 may be supported by a plurality of posts (e.g., two posts). The post 13 may also be referred to as a post portion.

The gantry 11 includes the opening 15 that forms an imaging space related to imaging of the subject P. The gantry 11 is a substantially cylindrical structure provided with the opening 15. As illustrated in FIG. 1, the gantry 11 accommodates an X-ray tube 17 and an X-ray detector 19 that are opposed to each other with respect to the opening 15. The X-ray tube 17 and the X-ray detector 19 are included in the imaging system related to imaging of the subject P according to the present embodiment.

The imaging system may further include data acquisition circuitry (hereinafter also referred to as a data acquisition system (DAS)) 33, a high-voltage generator 31, a collimator, and a wedge. In other words, the gantry 11 includes the imaging system related to imaging of the subject P. The gantry 11 is supported by the post 13 so that the gantry 11 can be moved in the vertical direction along the post 13.

The gantry 11 is supported by the post 13 so that the orientation of the opening 15 can be changed between the vertical direction and the horizontal direction. The orientation of the opening 15 corresponds to, for example, a direction in which the couchtop 30 is inserted in the opening 15, or a direction along a rotation axis A1.

The gantry 11 also includes a main frame (not illustrated) formed of metal such as aluminum, and a rotation frame 21 that is rotatably supported by the main frame about the rotation axis A1 via a bearing or the like. A contact portion between the main frame and the rotation frame 21 is provided with an annular electrode (not illustrated). A conductive slider (not illustrated) is attached to the contact portion of the main frame so that the slider can be brought into sliding contact with the annular electrode.

The post 13 is a substrate that supports the gantry 11 separately from a floor surface.

The post 13 has a columnar shape such as a cylindrical shape or a prismatic shape. The post 13 is formed of any material such as plastic or metal. The post 13 is attached to, for example, a side surface of the gantry 11. The post 13 supports the gantry 11 slidably in the vertical direction in a state where the rotation axis A1 of the opening 15 is substantially perpendicular to the floor surface so as to perform X-ray CT imaging of the subject P in a seated-position posture or a standing-position posture.

Typically, the post 13 is provided on one side of the gantry 11. However, the present embodiment is not limited to this example. For example, two posts 13 may be respectively connected to the both sides of the gantry 11. In other words, at least one post 13 supports the gantry 11 so as to be movable in the vertical direction.

An example where the post 13 has a columnar shape has been described above. However, the present embodiment is not limited to this example. For example, the post 13 may have any shape, such as a U-shape, as long as at least one side of the gantry 11 can be supported.

The post 13 supports the gantry 11 in such a manner that the rotation axis A1 can rotate about a horizontal axis (hereinafter also referred to as a tilt axis) that is parallel to the floor surface between the vertical direction and the horizontal direction. The post 13 and the gantry 11 are connected via, for example, a slewing ring bearing or the like so that the gantry 11 can be rotated about the tilt axis.

Specifically, the post 13 is provided with a linear guide along the vertical direction. A block configured to move along the linear guide is provided with a slewing ring bearing. The block is driven by a motor under the control of movement control circuitry 27, so that the block can move along the linear guide.

A gear fitted to a gear (internal teeth) in the slewing ring bearing is connected to a rotation shaft of the motor via various gears and the like that generate a predetermined torque. The internal teeth in the slewing ring bearing are rotated by being driven by the motor under the control of the movement control circuitry 27.

With the configurations described above, the gantry 11 can be rotated about the X-axis illustrated in FIG. 1 as a rotation axis and can be moved along the vertical direction. The above-described linear guide and slewing ring bearing correspond to a gantry movement mechanism 131 related to the movement of the gantry 11. In other words, the gantry movement mechanism 131 is mounted on the post 13.

The gantry movement mechanism 131 moves the block along the linear guide located along the vertical direction under the control of the movement control circuitry 27, thereby moving the gantry 11. This enables the gantry 11 to move up and down along the vertical direction. A mechanism related to the movement of the gantry 11 along the vertical direction is not limited to a linear guide or the like, but instead may be implemented by an existing mechanism such as a rack and pinion.

The gantry movement mechanism 131 causes the gantry 11 to rotate between the horizontal direction and the vertical direction by the rotation of the internal teeth in the slewing ring bearing under the control of the movement control circuitry 27.

A rotation mechanism that causes the gantry 11 to rotate is not limited to a slewing ring bearing, but instead may be implemented by an existing mechanism. The rotation of the gantry 11 by the rotation mechanism enables switching between a standing-position or seated-position imaging state (also referred to as a standing-position mode) and a lying-position imaging state (also referred to as a lying-position mode), or switching between the standing-position mode and the lying-position mode.

For example, in the case of performing lying-position imaging on the subject P, the gantry movement mechanism 131 causes the gantry 11 to rotate so that the opening 15 is oriented in the vertical direction under the control of the movement control circuitry 27. After the subject P lies down on a couchtop 30, couchtop movement mechanisms 37 to be described below move the couchtop 30 horizontally, thereby enabling lying-position imaging of the subject P, like in the general X-ray CT apparatus.

In the case of performing standing-position imaging on the subject P, the rotation mechanism in the gantry movement mechanism 131 causes the gantry 11 to rotate so that the opening 15 is oriented in the horizontal direction under the control of the movement control circuitry 27. While the subject P stands upright with his or her back leaning against the post 13, standing-position imaging is executed by moving the gantry 11 up and down. In the standing-position mode, the couchtop movement mechanisms 37 to be described below cause the couchtop 30 to evacuate to a position where the couchtop 30 does not interfere with the gantry 11 under the control of the movement control circuitry 27.

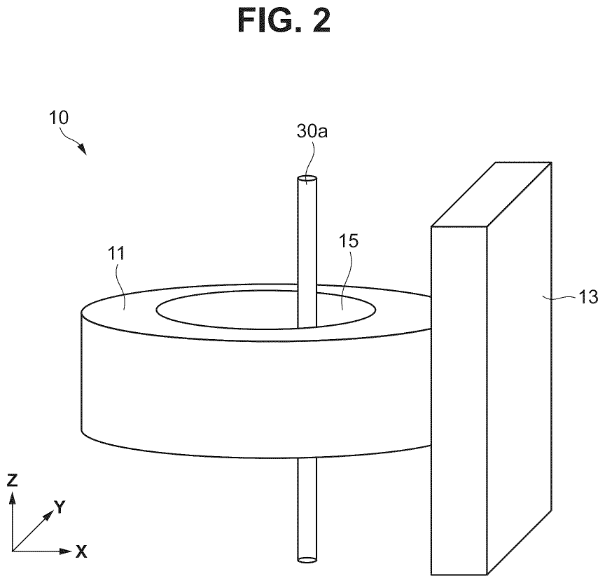

FIG. 2 is a perspective view illustrating an example of the state of the gantry apparatus 10 in the standing-position mode. As illustrated in FIG. 2, according to the present embodiment, in the standing-position mode, standing-position imaging of the subject P is performed in a state where a subject fixing pole 30a is present in the gantry 11. The subject P stands upright with his or her back slightly leaning against the subject fixing pole 30a, so that a stable standing position posture can be maintained without fluctuations.

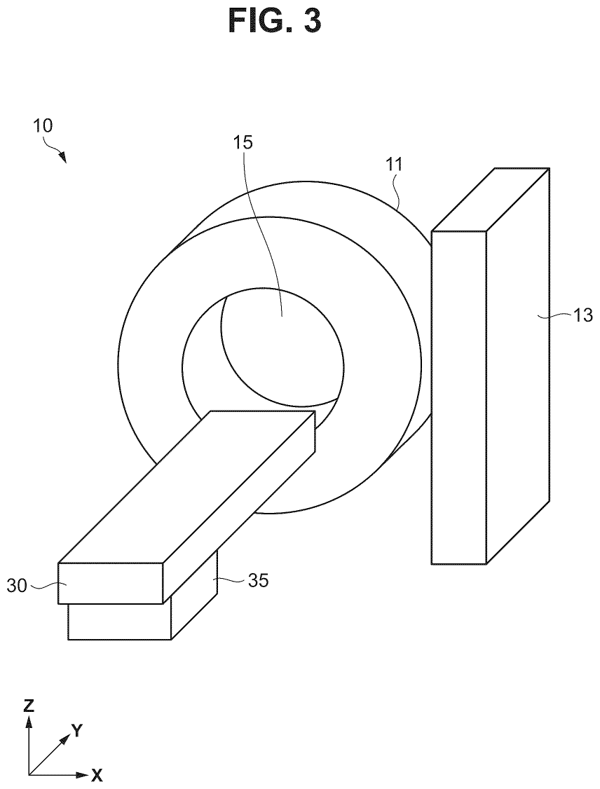

FIG. 3 is a perspective view illustrating an example of the state of the gantry apparatus 10 in the lying-position mode. As illustrated in FIG. 3, in the lying-position mode, the couchtop 30 is supported by a base 35 via the couchtop movement mechanisms 37 in a horizontal state. In this case, the couchtop 30 is freely movable along the major axis direction of the couchtop 30 under the control of the movement control circuitry 27.

The X-ray tube 17 is a vacuum tube that generates X-rays by radiating thermo-electrons from a cathode (filament) to an anode (target) by application of a high voltage from the high-voltage generator 31 and supply of a filament current. X-rays are generated due to collision of thermo-electrons on the target. The X-rays generated at the focal point of the tube in the X-ray tube 17 are formed in, for example, a cone beam shape via the collimator and are emitted to the subject P.

Examples of the X-ray tube 17 include a rotating anode X-ray tube that generates X-rays by emitting thermo-electrons onto a rotating anode. In the present embodiment, the X-ray tube 17 is also applicable to a single-tube X-ray CT apparatus and a so-called multi-tube X-ray CT apparatus having a configuration in which a plurality of pairs of X-ray tubes 17 and X-ray detectors 19 is mounted on the rotation frame 21.

The X-ray detector 19 detects an X-ray that is emitted from the X-ray tube 17 and has passed through the subject P, and outputs an electric signal corresponding to the amount of the X-ray to the DAS 33. The X-ray detector 19 includes, for example, a plurality of detection element arrays in which detection elements are arranged along a single arc about the focal point of the X-ray tube 17 in a channel direction. The X-ray detector 19 has a structure in which, for example, the plurality of detection element arrays is arranged in a slice direction (a column direction or a row direction).

Various types of the X-ray CT apparatus 1, including a rotate/rotate-type (third generation CT) having a configuration in which the X-ray tube 17 and the X-ray detector 19 rotate together around the subject P, and a stationary/rotate-type (fourth generation CT) having a configuration in which a large number of X-ray detection elements arrayed in a ring shape are fixed and the X-ray tube 17 alone rotates around the subject P, are applicable to the present embodiment. For the sake of specific explanation, a third generation CT will be described below as an example of the X-ray CT apparatus 1 according to the present embodiment.

The X-ray detector 19 is an indirect-conversion detector including a grid, a scintillator array, and an optical sensor array. The scintillator array includes a plurality of scintillators, and each scintillator includes a scintillator crystal that outputs light having a quantity of photon corresponding to an amount of incident X-rays. The grid is located on an X-ray incident side of the scintillator array and includes an X-ray shield plate having a function of absorbing scattered X-rays.

The grid may be referred to as a collimator (one-dimensional collimator or two-dimensional collimator). The optical sensor array has a function of converting an amount of light from the scintillators into a corresponding electric signal, and includes, for example, an optical sensor such as a photo multiplier (PMT).

The X-ray detector 19 may be a direct-conversion detector including a semiconductor element that converts an incident X-ray into an electric signal. Alternatively, the X-ray detector 19 may be a photon counting X-ray detector.

The rotation frame 21 includes the opening 15. The X-ray tube 17 that generates X-rays is attached to the rotation frame 21. Specifically, the rotation frame 21 is an annular frame that supports the X-ray tube 17 and the X-ray detector 19 in opposing positions to rotate the X-ray tube 17 and the X-ray detector 19 by the gantry control apparatus 25 to be described below.

The rotation frame 21 is supported rotatably about the main frame via a support bearing. The rotation frame 21 receives power from the rotation drive apparatus 23 under the control of the gantry control apparatus 25, and is rotated about the rotation axis A1 at a constant angular velocity about the rotation axis A1.

The rotation frame 21 includes not only the X-ray tube 17 and the X-ray detector 19, but also includes and supports the high-voltage generator 31 and the DAS 33. The rotation frame 21 having a configuration as described above is accommodated in a substantially cylindrical housing in which the opening 15 that forms the imaging space is formed. A central axis of the opening 15 coincides with the rotation axis A1 of the rotation frame 21.

Detected data generated by the DAS 33 is transmitted to, for example, a receiver that includes a photodiode and is provided on a non-rotational part (e.g., main frame) of the gantry apparatus 10 via optical communication from a transmitter including a light-emitting diode (LED), and is transferred to the console apparatus 100.

The method of transmitting detected data to the non-rotational part of the gantry apparatus 10 from the rotation frame 21 is not limited to the above-described optical communication, and any non-contact data transfer method may be employed.

The rotation drive apparatus 23 generates power for rotating the rotation frame 21 according to a control operation from the gantry control apparatus 25. The rotation drive apparatus 23 performs a driving operation at a rotational speed corresponding to a duty ratio or the like of a drive signal from the gantry control apparatus 25, thereby generating power. The rotation drive apparatus 23 is implemented by, for example, a motor such as a direct drive motor or a servo motor. The rotation drive apparatus 23 is accommodated in, for example, the gantry 11.

The gantry control apparatus 25 controls the high-voltage generator 31, the rotation drive apparatus 23, the movement control circuitry 27, and the DAS 33 according to a command from the console apparatus 100. The gantry control apparatus 25 has a function of receiving an input signal from an input interface attached to the console apparatus 100 or the gantry apparatus 10 and controlling the operation of the gantry apparatus 10.

For example, the gantry control apparatus 25 receives an input signal and performs a control operation for rotating the rotation frame 21, a control operation for tilting the gantry apparatus 10, and the like. The gantry control apparatus 25 may be provided on the post 13 in the gantry apparatus 10, or may be provided on the console apparatus 100. Each function to be implemented by the gantry control apparatus 25 may be implemented as a gantry control function in processing circuitry 107 in the console apparatus 100.

The gantry control apparatus 25 includes, as hardware resources, processing devices (processors), such as a central processing unit (CPU) and a micro processing unit (MPU), and storage devices (memories) such as a read only memory (ROM) and a random access memory (RAM).

The gantry control apparatus 25 may also be implemented by an application specific integrated circuit (ASIC), a field programmable gate array (FPGA), and any other complex programmable logic device (CPLD) or simple programmable logic device (SPLD).

The processing device reads out programs stored in the storage device and executes the programs, thereby implementing the functions described above. Instead of storing programs in the storage device, programs may be directly incorporated in the circuitry of the processing device. In this case, the processing device reads out programs incorporated in the circuitry and executes the programs, thereby implementing the functions described above.

The subject P can be placed on the couchtop 30 in the lying-position mode, and the couchtop 30 can be inserted into the opening 15. The couchtop 30 is supported on the base 35 via the couchtop movement mechanisms 37. Specifically, the couchtop 30 is held by the couchtop movement mechanisms 37 that are provided at the both ends in the Y-axis direction of the base 35.

The couchtop movement mechanism 37 enables the couchtop 30 to move along the direction in which the opening 15 penetrates. In other words, the couchtop 30 is fixed so as to be slidable and movable relative to the gantry 11 along the rotation axis A1 of the rotation frame 21 in the imaging system via the couchtop movement mechanism 37.

The couchtop movement mechanism 37 moves the couchtop 30 under the control of the movement control circuitry 27.

The couchtop movement mechanism 37 is composed of, for example, a roller guide or the like. The couchtop movement mechanism 37 can be implemented by frictional driving or a configuration such as a belt mechanism. The couchtop movement mechanism 37 is not limited to a roller guide, frictional driving, a belt mechanism, or the like, but instead may be implemented by an existing mechanism as appropriate.

The couchtop movement mechanism 37 may be mounted on an up-and-down motion mechanism. The up-and-down motion mechanism is mounted on, for example, the couchtop movement mechanism 37 and is provided on the base 35. The up-and-down motion mechanism can move the couchtop 30 in a direction perpendicular to a surface on which the subject P is placed on the couchtop 30.

For example, the up-and-down motion mechanism can be implemented by an actuator (e.g., piston type) configured to move (push up) the rotation shaft of the roller guide along the Y-axis direction. The up-and-down motion mechanism is not limited to an actuator, but instead may be implemented any other mechanism.

A left-to-right motion mechanism may be provided between the couchtop movement mechanism 37 and the couchtop 30. For example, couchtop support members that cover the lower surface and the side surface of the couchtop 30 are provided on the lower surface of the couchtop 30 and the side surface of the couchtop 30. The left-to-right motion mechanism includes a block, a ball screw, a motor, and a belt. The ball screw stretches along the minor axis direction of the couchtop 30. The block is attached to the ball screw.

The block is connected to couchtop support members. A torque from the motor is transmitted to the ball spring via the belt. When the motor is rotated under the control of the movement control circuitry 27, the torque from the motor is transmitted to the ball screw. This allows the ball screw to rotate. Along with the rotation of the ball screw, the block moves along the minor axis direction of the couchtop 30.

The movement control circuitry 27 controls the movement of each of the gantry 11 and the couchtop 30.

For example, if an instruction to capture an image of the subject P in the standing-position mode is issued from a user, the movement control circuitry 27 controls the gantry movement mechanism 131 to rotate the gantry 11 so that the opening 15 is oriented in the vertical direction.

For example, if an instruction to capture an image of the subject P in the standing-position mode is issued from the user, the movement control circuitry 27 controls the couchtop movement mechanism 37 to evacuate the couchtop 30 to a position where the couchtop 30 does not interfere with the gantry 11 even when the gantry 11 is moved along the vertical direction.

The base 35 may be provided with a movement mechanism such as a caster. In this case, the base 35 may be manually moved by the user to evacuate the couchtop 30, or the base 35 may be automatically moved by a drive source such as a motor to evacuate the couchtop 30.

The movement control circuitry 27 can be implemented by a processor or the like as described above.

While FIG. 1 illustrates an example where the movement control circuitry 27 is mounted on the post 13, the movement control circuitry 27 may be mounted on the gantry 11 or may be mounted on the console apparatus 100. Each function to be implemented by the movement control circuitry 27 may be mounted on the processing circuitry 107 or may be mounted on the gantry control apparatus 25 as a movement control function.

An operation panel 29 is implemented by a switch button, a touch pad for performing an input operation by touching on an operation surface, a touch panel display having a configuration in which a display screen and a touch pad are integrated together, or the like. The operation panel 29 converts an input operation received from the user into an electric signal, and outputs the electric signal to the gantry control apparatus 25.

The operation panel 29 receives a selection operation of selecting an imaging protocol including, for example, a standing-position mode related to imaging of the subject P in the standing position posture, a seated-position mode related to imaging of the subject P in the seated position posture, or a lying-position mode related to imaging of the subject P in the lying position posture. The operation panel 29 is provided on, for example, the post 13.

The high-voltage generator 31 includes electric circuitry such as a transformer and a rectifier, and generates a high voltage to be applied to the X-ray tube 17 and a filament current to be supplied to the X-ray tube 17. The high-voltage generator 31 controls an output voltage based on X-rays emitted from the X-ray tube 17. The high-voltage generator 31 may be a transformer type generator or an inverter type generator.

The high-voltage generator 31 may be provided on the rotation frame 21, or may be provided on the main frame of the gantry 11.

The wedge (not illustrated) is a filter for adjusting the amount of X-rays emitted from the X-ray tube 17. Specifically, the wedge is a filter that allows the X-rays emitted from the X-ray tube 17 to transmit therethrough for attenuation, so that the subject P is irradiated with the X-rays from the X-ray tube 17 in a predetermined distribution.

The wedge is, for example, a wedge filter or a bow-tie filter, and is formed by processing aluminum with a predetermined target angle and a predetermined thickness.

The collimator (not illustrated) includes a combination of a plurality of lead plates, which form a slit, to converge the X-rays having passed through the wedge in an irradiation range.

The DAS 33 includes an amplifier for performing amplification processing on an electric signal output from each X-ray detection element of the X-ray detector 19, and an analog-to-digital (A/D) converter for converting an electric signal into a digital signal, and generates detected data. The detected data generated by the DAS 33 is transferred to the console apparatus 100.

The console apparatus 100 includes a memory 101, a display 103, an input interface 105, and the processing circuitry 107. Data communication between the memory 101, the display 103, the input interface 105, and the processing circuitry 107 is performed via, for example, a bus.

The memory 101 is a storage device for storing various information, such as a hard disk drive (HDD), a solid state drive (SSD), or an integrated circuit storage device. For example, the memory 101 stores projection data and reconstruction image data.

For example, the memory 101 stores parameter information 1011. The parameter information 1011 is stored by associating a VolumeEC parameter with information indicating which one of the lying-position mode and the standing-position mode is set as an imaging mode. The parameter information 1011 is used for the processing circuitry 107 to be described below to perform VolumeEC parameter determination processing and X-ray irradiation conditions adjustment processing.

The memory 101 is not limited to an HDD, an SSD, or the like, but instead may be a drive apparatus configured to read and write various information from and to portable storage media such as a compact disc (CD), a digital versatile disc (DVD), and a flash memory, semiconductor memory elements such as a RAM, and the like.

A storage region of the memory 101 may be located within the console apparatus 100, or may be located within an external storage device connected via a network. The memory 101 stores control programs according to the present embodiment. The memory 101 stores volume data and the like generated by pre-scan or main-scan processing.

The display 103 displays various types of information. For example, the display 103 outputs medical images (CT images) generated by the processing circuitry 107, a graphical user interface (GUI) for receiving various operations from the user, and the like.

As the display 103, for example, a liquid crystal display (LCD), a cathode ray tube (CRT) display, an organic electro luminescence display (OELD), a plasma display, or any other display can be used as appropriate.

The display 103 may be provided on the gantry apparatus 10. The display 103 may be a desktop display, or may be composed of a tablet terminal or the like configured to establish wireless communication with the main body of the console apparatus 100. The display 103 corresponds to a display unit.

The input interface 105 receives various input operations from the user, converts the received input operations into electric signals, and outputs the electric signals to the processing circuitry 107. For example, the input interface 105 receives acquisition conditions for acquiring projection data, reconstruction conditions for reconstructing a CT image, image processing conditions for generating a post-processing image based on the CT image, and the like from the user.

As the input interface 105, for example, a mouse, a keyboard, a trackball, a switch, a button, a joystick, a touch pad, or a touch panel display can be used.

In the present embodiment, the input interface 105 is not limited to input interfaces including physical operation members such as a mouse, a keyboard, a trackball, a switch, a button, a joystick, a touch pad, and a touch panel display.

Examples of the input interface 105 also include processing circuitry that receives an electric signal corresponding to an input operation from an external input device provided separately from the apparatus and outputs the electric signal to processing circuitry 107.

The input interface 105 may be provided on the gantry apparatus 10. The input interface 105 may be composed of a tablet terminal or the like configured to establish wireless communication with the main body of the console apparatus 100. The input interface 105 corresponds to an input unit.

The processing circuitry 107 controls an overall operation of the X-ray CT apparatus 1 according to an electric signal corresponding to an input operation output from the input interface 105. For example, the processing circuitry 107 includes, as hardware resources, a processor such as a CPU, an MPU, or a graphics processing unit (GPU), and a memory such as a ROM or a RAM.

The processing circuitry 107 causes a processor that executes programs loaded into a memory to execute a system control function 111, a determination function 113, an adjustment function 115, a pre-processing function 117, a reconstruction function 119, and an image processing function 121.

In this case, the system control function 111 is an example of a reception unit. The determination function 113 is an example of each of an identification unit and a determination unit. The adjustment function 115 is an example of each of a calculation unit and an adjustment unit.

Each of the system control function 111, the determination function 113, the adjustment function 115, the pre-processing function 117, the reconstruction function 119, and the image processing function 121 need not necessarily be implemented by a single processing circuitry. Processing circuitry may be formed by combining a plurality of independent processors and each processor may execute programs to thereby implement the system control function 111, the determination function 113, the adjustment function 115, the pre-processing function 117, the reconstruction function 119, and the image processing function 121.

The system control function 111 controls each function of the processing circuitry 107 based on an input operation received from the user via the input interface 105.

Specifically, the system control function 111 reads out control programs stored in the memory 101 and loads the control programs into the memory in the processing circuitry 107, thereby controlling each unit of the X-ray CT apparatus 1 according to the loaded control programs. For example, the system control function 111 controls each function of the processing circuitry 107 based on an input operation received from the user via the input interface 105.

In the present embodiment, the input operation includes a selection input of an imaging protocol. The imaging protocol includes information indicating which one of the standing-position mode and the lying-position mode is set as the imaging mode.

The determination function 113 determines a VolumeEC parameter used for calculation processing related to VolumeEC based on a type of an object present on an X-ray tube path (between the X-ray tube 17 and the X-ray detector 19). The calculation processing related to VolumeEC is, for example, processing of calculating estimated values for the body thickness of the subject P, a CT value, and the like based on a scanogram of the subject P.

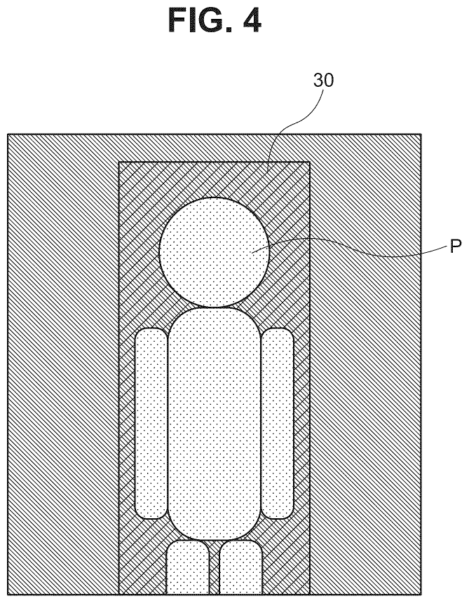

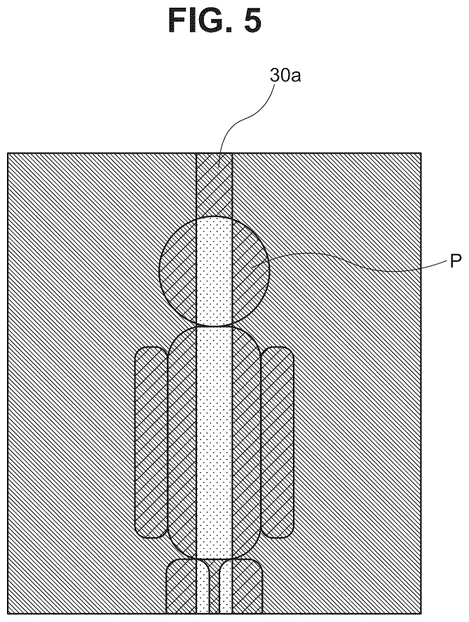

FIG. 4 illustrates an example of the scanogram of the subject P captured in the lying-position mode. FIG. 5 illustrates an example of the scanogram of the subject P captured in the standing-position mode. As illustrated in FIGS. 4 and 5, an object present on the X-ray tube path during imaging of the subject P in the lying-position mode is different from an object present on the X-ray tube path during imaging of the subject P in the standing-position mode, to be more specific, the couchtop 30 is present on the X-ray tube path during imaging of the subject P in the lying-position mode, while the subject fixing pole 30a is present on the X-ray tube path during imaging of the subject P in the standing-position mode.

For this reason, there may be a difference in the amount of absorption of X-rays on the subject P and there may also be a difference in the image quality of the scanogram. Accordingly, even when the body thickness of the subject P, the CT value, and the like are calculated using the same VolumeEC parameter, there may be a difference in the calculation result depending on which one of the standing-position mode and the lying-position mode is set as the imaging mode. In other words, there is a possibility that the body thickness of the subject P, the CT value, and the like cannot be accurately estimated and the X-ray irradiation conditions cannot be adjusted to optimum conditions depending on the imaging mode.

Therefore, the X-ray CT apparatus 1 according to the present embodiment stores an optimum VolumeEC parameter by taking into consideration an object present on the X-ray tube path for each imaging mode and calculates the body thickness of the subject P, the CT value, and the like using the VolumeEC parameter suitable for the imaging mode, thereby adjusting the X-ray irradiation conditions to optimum conditions.

For example, the determination function 113 identifies imaging conditions and also identifies which one of the standing-position mode and the lying-position mode is set as the imaging mode based on information included in the selected and input imaging protocol received by the system control function 111. The determination function 113 refers to the parameter information 1011 in the memory 101 and determines the VolumeEC parameter associated with the identified imaging mode to be the VolumeEC parameter used for calculation processing related to VolumeEC.

The adjustment function 115 performs calculation processing related to VolumeEC using the VolumeEC parameter determined by the determination function 113. The adjustment function 115 adjusts X-ray irradiation conditions based on the result of the calculation processing related to VolumeEC.

For example, the adjustment function 115 calculates the body thickness of the subject P, the CT value, and the like using the VolumeEC parameter determined by the determination function 113. The adjustment function 115 adjusts X-ray irradiation conditions (e.g., a tube voltage and a tube current) so that a CT image with a designated image quality can be generated based on the calculated body thickness of the subject P, the calculated CT value, and the like. In the present embodiment, information indicating the designated image quality is included in the imaging protocol.

The adjustment function 115 executes processing of adjusting the X-ray irradiation conditions in real time during imaging of the subject P. This configuration enables the X-ray CT apparatus 1 according to the present embodiment to perform calculation processing related to VolumeEC under optimum conditions and capture an image of the subject P using minimum X-ray irradiation conditions under which high image quality can be secured while reducing the exposure dose of the subject P and also reducing noise.

Referring again to FIG. 1, the description of the configuration of the X-ray CT apparatus 1 will be continued. The pre-processing function 117 generates data by performing pre-processing, such as logarithmic transformation processing, offset correction processing, inter-channel sensitivity correction processing, or beam hardening correction, on raw data output from the DAS 33. Data obtained before pre-processing is also referred to as raw data, and data obtained after pre-processing is also referred to as projection data.

The reconstruction function 119 executes reconstruction processing on projection data, thereby generating a reconstruction image.

For example, the reconstruction function 119 performs reconstruction processing using a filtered back projection (FBP) method, a successive approximation reconstruction method, or the like on the projection data generated by the pre-processing function 117, thereby generating CT image data. In other words, the reconstruction function 119 generates a reconstruction image based on an output from the imaging system. The reconstruction function 119 stores the generated reconstruction image data in the memory 101.

The image processing function 121 performs various types of image processing on the reconstruction image generated by the reconstruction function 119. For example, the image processing function 121 generates a display image by performing three-dimensional image processing, such as volume rendering, surface volume rendering, image value projection processing, multi-planer reconstruction (MPR) processing, or curved MPR (CPR) processing, on the CT image.

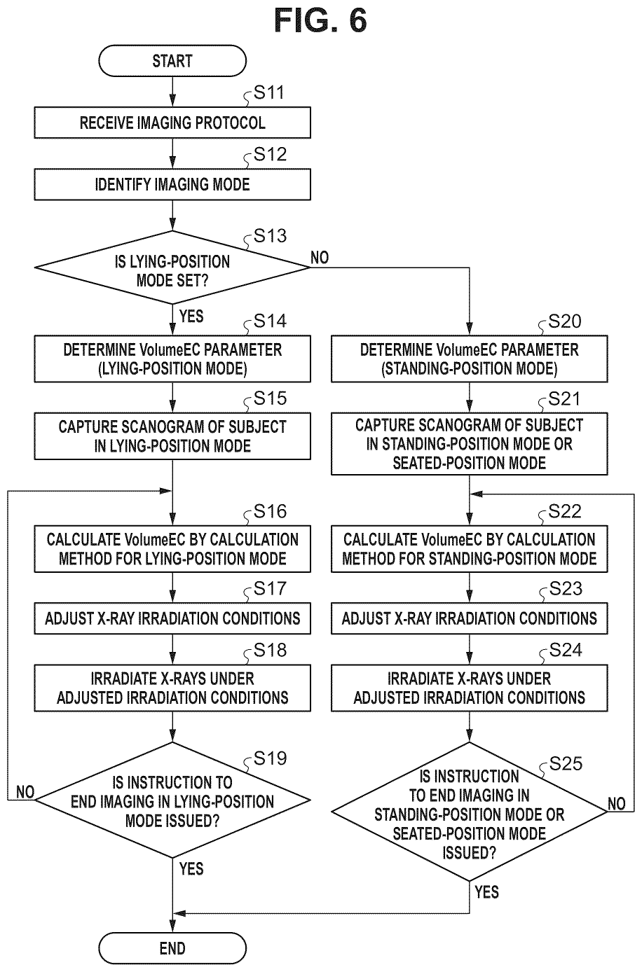

Processing to be executed by the X-ray CT apparatus 1 according to the present embodiment will be described below. FIG. 6 is a flowchart illustrating an example of processing to be executed by the X-ray CT apparatus 1 according to the embodiment.

First, in step S11, the system control function 111 receives a selection input of an imaging protocol from the user. The imaging protocol includes information indicating which one of the lying-position mode and the standing-position mode is set as the imaging mode.

Next, in step S12, the determination function 113 identifies the imaging mode for the subject P. For example, the determination function 113 identifies the imaging mode for the subject P based on the information indicating which one of the lying-position mode and the standing-position mode is set as the imaging mode included in the imaging protocol received in step S11. Next, in step S13, the determination function 113 determines whether the identified imaging mode is the lying-position mode (standing-position mode).

If the lying-position mode is set as the imaging mode (YES in step S13), the processing proceeds to step S14. In step S14, the determination function 113 refers to the parameter information 1011 in the memory 101 and determines the VolumeEC parameter associated with the lying-position mode to be the VolumeEC parameter used for calculation processing related to VolumeEC.

Next, in step S15, the system control function 111 captures a scanogram of the subject P in the lying-position mode. For example, the system control function 111 controls each unit of the X-ray CT apparatus 1 according to a user operation to capture a scanogram of the subject P. The scanogram of the subject P captured in the lying-position mode may be acquired in advance.

Next, in step S16, the adjustment function 115 performs calculation processing related to VolumeEC using the VolumeEC parameter for the lying-position mode. For example, the adjustment function 115 calculates the body thickness of the subject P, the CT value, and the like using the VolumeEC parameter for the lying-position mode determined in step S14.

Next, in step S17, the adjustment function 115 adjusts the X-ray irradiation conditions. For example, the adjustment function 115 adjusts X-ray irradiation conditions so that the CT image with the image quality corresponding to information indicating the designated image quality included in the imaging protocol received in step S11 can be generated based on the body thickness of the subject P, the CT value, and the like calculated in step S16.

Next, instep S18, the system control function 111 irradiates the subject P with X-rays under the adjusted X-ray irradiation conditions. For example, the system control function 111 controls each unit of the X-ray CT apparatus 1 to irradiate the subject P with X-rays under the X-ray irradiation conditions adjusted in step S17.

Next, in step S19, the system control function 111 determines whether an instruction to end imaging of the subject P in the lying-position mode is issued. If the instruction to end imaging of the subject P in the lying-position mode is not issued (NO in step S19), the processing returns to step S16. On the other hand, if the instruction to end imaging of the subject P in the lying-position mode is issued (YES in step S19), the processing of this flowchart ends.

On the other hand, if the standing-position mode or the seated-position mode is set as the imaging mode (NO in step S13), the processing proceeds to step S20. In step S20, the determination function 113 refers to the parameter information 1011 in the memory 101 and determines the VolumeEC parameter associated with the standing-position mode to be the VolumeEC parameter used for calculation processing related to VolumeEC.

Next, in step S21, the system control function 111 captures a scanogram of the subject P in the standing-position mode or the seated-position mode. For example, the system control function 111 controls each unit of the X-ray CT apparatus 1 according to a user operation to capture a scanogram of the subject P. The scanogram of the subject P captured in the standing-position mode may be acquired in advance.

Next, in step S22, the adjustment function 115 performs calculation processing related to VolumeEC using the VolumeEC parameter for the standing-position mode. For example, the adjustment function 115 calculates the body thickness of the subject P, the CT value, and the like using the VolumeEC parameter for the standing-position mode determined in step S20.

Next, in step S23, the adjustment function 115 adjusts the X-ray irradiation conditions. For example, the adjustment function 115 adjusts the X-ray irradiation conditions so that the CT image with the image quality corresponding to information indicating the designated image quality included in the imaging protocol received in step S11 can be generated based on the body thickness of the subject P, the CT value, and the like calculated in step S22.

Next, in step S24, the system control function 111 irradiates the subject P with X-rays under the adjusted X-ray irradiation conditions. For example, the system control function 111 controls each unit of the X-ray CT apparatus 1 to irradiate the subject P with X-rays under the X-ray irradiation conditions adjusted in step S23.

Next, in step S25, the system control function 111 determines whether an instruction to end imaging of the subject P in the standing-position mode or the seated-position mode is issued. If the instruction to end imaging of the subject P in the standing-position mode or the seated-position mode is not issued (NO in step S25), the processing returns to step S22. On the other hand, if the instruction to end imaging of the subject P in the standing-position mode or the seated-position mode is issued (YES in step S25), the processing of this flowchart ends.

As described above, the X-ray CT apparatus 1 according to the present embodiment identifies the posture of the gantry 11 and the object present on the X-ray path, determines the VolumeEC parameter used for calculation processing related to VolumeEC based on the identified posture of the gantry 11, the identified object present on the X-ray path, and the parameter information 1011 to be stored by associating the posture of the gantry 11, the object present on the X-ray path, and the VolumeEC parameter for calculation processing related to VolumeEC, performs calculation processing related to VolumeEC using the determined VolumeEC parameter, and adjusts the X-ray irradiation conditions based on the result of the calculation processing related to VolumeEC.

For example, if calculation processing related to VolumeEC is performed using the same VolumeEC parameter regardless of the state of the gantry 11, there is a difference in the object (the couchtop 30 or the subject fixing pole 30a) present on the X-ray path and there is also a difference in the amount of absorption of X-rays depending on the object, which may cause a difference in the image quality of the scanogram of the subject P depending on the state of the gantry 11. This may cause a difference in the result of calculation processing related to VolumeEC even when the calculation processing (calculation of the body thickness of the subject P, the CT value, and the like) related to VolumeEC is performed using the same VolumeEC parameter. In other words, there is a possibility that the calculation processing related to VolumeEC cannot be accurately performed and the X-ray irradiation conditions cannot be adjusted to optimum conditions depending on the state of the gantry 11. On the other hand, the X-ray CT apparatus 1 according to the present embodiment can perform calculation processing related to VolumeEC using the VolumeEC parameter suitable for each type of the object present on the X-ray path. This configuration enables the X-ray CT apparatus 1 according to the present embodiment to perform calculation processing related to VolumeEC under optimum conditions depending on the state of the gantry 11 (type of the object present on the X-ray path).

The above-described embodiment can be modified as appropriate by changing a part of the configuration of the X-ray CT apparatus 1 or some of the functions of the X-ray CT apparatus 1. Accordingly, modified examples of the above-described embodiment will be described below as other embodiments. In the following description, differences from the above-described embodiment will be mainly described, and detailed descriptions of the same contents as those described above will be omitted. The following modified examples may be individually carried out, or may be carried out in combination as appropriate.

### Modified Example 1

The embodiment described above illustrates a configuration in which if the seated position is set as the imaging posture of the subject P, the VolumeEC parameter is determined and calculation processing related to VolumeEC is executed, assuming that the standing-position mode is set as the imaging mode, like in a case where the standing position is set as the imaging posture of the subject P. In Modified Example 1, a configuration in which the VolumeEC parameter in the seated-position imaging state (also referred to as the seated-position mode) is stored and calculation processing related to VolumeEC is executed using the VolumeEC parameter corresponding to the seated-position mode when the seated position is set as the imaging posture of the subject P will be described.

In Modified Example 1, the parameter information 1011 in the memory 101 is stored by associating information indicating the seated-position mode as the imaging mode with an optimum VolumeEC parameter for the seated-position mode.

In Modified Example 1, the imaging protocol includes information indicating which one of the lying-position mode, the standing-position mode, and the seated-position mode is set as the imaging mode.

If the imaging mode included in the imaging protocol received by the system control function 111 is the seated-position mode, the determination function 113 refers to the parameter information 1011 in the memory 101 and determines the VolumeEC parameter associated with the seated-position mode to be the VolumeEC parameter used for calculation processing related to VolumeEC.

In Modified Example 1, if the gantry 11 is in the seated position state, the VolumeEC parameter that is suitable for the seated-position mode and is different from the VolumeEC parameter corresponding to the standing-position mode can be used for calculation processing related to VolumeEC. In other words, according to Modified Example 1, calculation processing related to VolumeEC can be performed under more suitable conditions.

### Modified Example 2

The embodiment described above illustrates a configuration in which the imaging posture is one of the lying position (an installation surface of the post 13 is parallel to the axis of the body of the subject P), the standing position, and the seated position (the installation surface of the post 13 is vertical to the axis of the body of the subject P). In Modified Example 2, a configuration in which an image of the subject P can be captured by setting an angle formed between the installation surface of the post 13 and the axis of the body of the subject P to a desired angle within a range from 90° (the installation surface of the post 13 is vertical to the axis of the body of the subject P) to 180° (the installation surface of the post 13 is parallel to the axis of the body of the subject P) will be described.

In Modified Example 2, the couchtop movement mechanism 37 includes a mechanism configured to change the angle formed between the surface on which the subject P is placed on the couchtop 30 and the installation surface of the post 13 to a desired angle within the range from 90° to 180°. The system control function 111 according to Modified Example 2 receives an input operation for inputting a desired angle of the couchtop 30 (also referred to as a couchtop angle) with respect to the installation surface of the post 13 from the user. For example, the system control function 111 receives an input operation for inputting a desired angle within the range from 90° to 180° in units of 5°.

The movement control circuitry 27 according to Modified Example 2 performs a control operation for the user to change the angle of the couchtop 30 (hereinafter also referred to as the couchtop angle) with respect to the installation surface of the post 13 to a desired angle within the range from 90° to 180°.

When the user changes the angle of the couchtop 30 to a desired angle within the range from 90° to 180°, the movement control circuitry 27 causes the gantry 11 to rotate so that the opening 15 can be vertical to the surface on which the subject P is placed on the couchtop 30.

In Modified Example 2, the parameter information 1011 in the memory 101 is stored by associating an optimum VolumeEC parameter with an angle of the couchtop 30 for each angle of the couchtop 30 (every 5° within the range from 90° to 180° in Modified Example 2).

In Modified Example 2, the imaging protocol includes information indicating the angle of the couchtop 30 at which an image of the subject P is captured.

The determination function 113 refers to the parameter information 1011 in the memory 101 and determines the VolumeEC parameter associated with information indicating the angle of the couchtop 30 included in the imaging protocol received by the system control function 111 to be the VolumeEC parameter used for calculation processing related to VolumeEC.

In Modified Example 2, the VolumeEC parameter suitable for each angle of the couchtop 30 can be used for calculation processing related to VolumeEC. In other words, according to Modified Example 2, calculation processing related to VolumeEC can be performed under more suitable conditions.

### Modified Example 3

The embodiment described above illustrates a configuration in which an optimum VolumeEC parameter for each imaging mode is determined in advance for each imaging mode. In Modified Example 3, a configuration in which the same VolumeEC parameter is used regardless of the imaging mode and an optimum calculation method (for example, multiplying a correction coefficient prepared for each imaging mode after calculation processing related to VolumeEC) for each imaging mode is determined for each imaging mode will be described.

In Modified Example 3, one VolumeEC parameter is stored in the parameter information 1011 in the memory 101. The memory 101 further stores calculation information. The calculation information is stored by associating information indicating the imaging mode with the correction coefficient. In this case, the correction coefficient is determined by taking into consideration the type of the object (the couchtop 30 or the subject fixing pole 30a) present on the X-ray tube path.

In Modified Example 3, assume that the system control function 111 executes storage processing by associating information indicating the imaging mode with the correction coefficient. In this case, the system control function 111 is an example of a storage control unit.

In Modified Example 3, the adjustment function 115 identifies the imaging mode for capturing an image of the subject P based on information included in the imaging protocol received by the system control function 111. The adjustment function 115 refers to the calculation information in the memory 101 and identifies the correction coefficient associated with the identified imaging mode.

The adjustment function 115 performs calculation processing related to VolumeEC using the VolumeEC parameter determined by the determination function 113, and uses the value obtained by multiplying the calculation result by the identified correction coefficient as the result of the calculation processing related to VolumeEC.

According to Modified Example 3, there is no need to prepare a VolumeEC parameter for each imaging mode, which leads to a reduction in the burden of work for preparing the VolumeEC parameter.

### Modified Example 4

The embodiment described above illustrates a configuration in which the type of the object present on the X-ray tube path is the couchtop 30 or the subject fixing pole 30a. In Modified Example 4, a configuration in which an image of the subject P is captured in a state where an object other than the couchtop 30 or the subject fixing pole 30a is inserted into the opening 15 of the gantry 11 will be described.

In Modified Example 4, during imaging of the subject P in the lying-position mode, not only the couchtop 30 (CT couch), but also an angio couch, a flat couchtop, and an operating table are used. These are examples of the type of the couch used for imaging of the subject P in the lying-position mode. Any other type of couch (couchtop) may also be used for imaging of the subject P.

In Modified Example 4, the parameter information 1011 in the memory 101 is stored by associating the imaging conditions, information indicating the object present on the X-ray tube path, and the VolumeEC parameter.

In Modified Example 4, the imaging protocol includes information indicating the object present on the X-ray tube path. Information indicating the type of the object present on the X-ray tube path is an example of each of object information and gantry state information.

The determination function 113 identifies imaging conditions and information indicating the object present on the X-ray tube path based on information included in the imaging protocol received by the system control function 111.

The determination function 113 refers to the parameter information 1011 in the memory 101 and determines the VolumeEC parameter associated with the identified information indicating the object present on the X-ray tube path to be the VolumeEC parameter used for calculation processing related to VolumeEC.

A plurality of objects may be present on the X-ray tube path. The objects present on the X-ray tube path may also include clothes worn by the subject P and drip tubing.

In Modified Example 4, even in a case where an image of the subject P is captured in a state where an object other than the couchtop 30 or the subject fixing pole 30a is inserted into the opening 15 of the gantry 11, the VolumeEC parameter suitable for the imaging mode can be used can be used for calculation processing related to VolumeEC. In other words, according to Modified Example 4, it is possible to perform calculation processing related to VolumeEC under conditions suitable for the state of the gantry 11.

According to at least one of the embodiments, modified examples, and the like described above, it is possible to perform calculation processing related to VolumeEC under conditions suitable for a gantry state in an X-ray CT apparatus configured to capture an image of a subject in at least the lying position state and the standing position state.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography (CT) apparatus comprising processing circuitry configured to:
identify gantry state information indicating a state of a gantry including an opening, the gantry state information including at least one of posture information indicating a posture of the gantry and object information indicating an object other than a subject present in the opening;
determine a VolumeEC parameter used for calculation processing related to VolumeEC, which is a function to adjust X-ray irradiation conditions for the subject, based on the identified gantry state information and parameter information to be stored by associating the gantry state information with the VolumeEC parameter for calculation processing related to the VolumeEC;
perform calculation processing related to the VolumeEC using the determined VolumeEC parameter; and
adjust the X-ray irradiation conditions based on a result of the calculation processing related to the VolumeEC.

2. The X-ray CT apparatus according to claim 1,
wherein the gantry state information includes the object information, and
wherein the processing circuitry is further configured to store the parameter information by associating the object information indicating a type of the object with the VolumeEC parameter for each type of the object, at least a part of the VolumeEC parameter being different in each type of the object.

3. The X-ray CT apparatus according to claim 2, wherein the object includes a couchtop on which the subject in a lying position state is placed during imaging, and a columnar subject holder configured to support the subject in a standing position state during imaging.

4. The X-ray CT apparatus according to claim 3, wherein the processing circuitry is configured to perform one of processing of: determining a calculation method for performing calculation processing related to VolumeEC using the VolumeEC parameter associated with another object including the subject holder based on the VolumeEC parameter associated with the couchtop and storing the parameter information by associating the calculation method with information indicating the type of the object, and
determining a calculation method for performing calculation processing related to VolumeEC using the VolumeEC parameter associated with another object including the couchtop based on the VolumeEC parameter associated with the subject holder and storing the parameter information by associating the calculation method with information indicating the type of the object.

5. The X-ray CT apparatus according to claim 3,
wherein the X-ray CT apparatus is configured to perform Angio-CT imaging on the subject, and
wherein the object further includes an angio couch on which the subject is placed during Angio-CT imaging of the subject.

6. The X-ray CT apparatus according to claim 1, wherein the processing circuitry is further configured to:
receive an input of at least imaging conditions for imaging of the subject and an imaging protocol including the gantry state information, and
identify the gantry state information based on the received imaging protocol.

7. An information processing method for adjusting X-ray irradiation conditions for an X-ray CT apparatus, the information processing method comprising:
identifying gantry state information indicating a state of a gantry including an opening, the gantry state information including at least one of posture information indicating a posture of the gantry and object information indicating an object other than a subject present in the opening;
determining a VolumeEC parameter used for calculation processing related to VolumeEC, which is a function to adjust X-ray irradiation conditions for the subject, based on the identified gantry state information and parameter information to be stored by associating the gantry state information with the VolumeEC parameter for calculation processing related to the VolumeEC;
performing calculation processing related to the VolumeEC using the determined VolumeEC parameter; and
adjusting the X-ray irradiation conditions based on a result of the calculation processing related to the VolumeEC.
